SEQUENCE LISTING (90 sequences)

SEQ ID NO: 1 - DNA, 150 bp, Human papilloma virus
cagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtattt

SEQ ID NO: 2 - DNA, 150 bp, Human papilloma virus
aacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgc

SEQ ID NO: 3 - DNA, 150 bp, Human papilloma virus
tagtgatactggggaggatatggttgactttattgacaattgtaatgtatacaacaatcaggcagaagcagagacagcacaggcattgtttcatgcacaggaagcggaggaacatgcagaggctgtgcaggttctaaaacgaaagtatgt

SEQ ID NO: 4 - DNA, 150 bp, Human papilloma virus
agatgacagtggcacggatttactagagtttatagatgattctatggaaaatagtatacaggcagacacagaggcagcccgggcattgtttaatatacaggaaggggaggatgatttaaatgctgtgtgtgcactaaaacgaaagtttgc

SEQ ID NO: 5 - DNA, 150 bp, Human papilloma virus
ctgtgacaggggggaggatatggtggactttataaatgatacagatatattaaacatacaggcagaaacagagacagcacaagcattatttcatgcacaggaggagcaaacacacaaagaggctgtacaggtcctaaaacgaaagtatgc

SEQ ID NO: 6 - DNA, 150 bp, Human papilloma virus
aacagatacaggttcagacctggcagactttattgatgattccacagatatttgtgtacaggcagagcgtgagacagcacaggtacttttacatatgcaagaggcccaaagggatgcacaagcagtgcgtgccttaaaacgaaagtatac

SEQ ID NO: 7 - DNA, 150 bp, Human papilloma virus
aacagatacagggtcggatatggtagattttattgacacacaattatccatttgtgaacaggcagagcaagagacagcacaggcattgttccatgcgcaggaagttcagaatgatgcacaggtgttgcatcttttaaaacgaaagtttgc

SEQ ID NO: 8 - DNA, 150 bp, Human papilloma virus
agatgatacaggatctgatttaataaactttatagatagtgaaactagtatttgcagtcaggcggaacaggagacagcacgggcgttgtttcaggcccaagaattacaggcaaacaaagaggctgtgcatcagttaaaacgaaagtttct

SEQ ID NO: 9 - DNA, 150 bp, Human papilloma virus
atatgatagtggaacagatctaatagattttatagatgattcaaatataaataatgaacaggcagaacatgaggcagcccgggcattgtttaatgcacaggaaggggaggatgatttacatgctgtgtctgcagtaaaacgaaagtttac

SEQ ID NO: 10 - DNA, 149 bp, Human papilloma virus
ggatgaaatagatacagatttagatggatttatagacgattcatatatacaaaatatacaggcagacgcagaaacagtcaacaattgttgcaagtacaaacagcacatgcagataaacagacgttgcaaaaactaaaacgaaagtatat

SEQ ID NO: 11 - DNA, 150 bp, Human papilloma virus
agacgatagtggtacagatttaatagagtttatagatgattcagtacaaagtactacacaggcagaagcagaggcagcccgagcgttgtttaatgtacaggaaggggtggacgatataaatgctgtgtgtgcactaaaacgaaagtttgc

SEQ ID NO: 12 - DNA, 149 bp, Human papilloma virus
aacagatacaggttcagacttggtagattttattgatgataccacaacaatttgtgtacaggcagagcgcgagacagcacaggccttgtttaatgtgcaggaagcccaaagggatgcacgggaaatgcatgttttaaaacgaaagtttg

SEQ ID NO: 13 - DNA, 150 bp, Human papilloma virus
aaatgatacagggtctgatataatagactttatagatacaaataacagtatttgcagtcaggcggaacaagagacagcacgggcgttgtttcaggtccaagaaacacaggcacacaaagaggctgcacagcatctaaaacgaaagttttt

SEQ ID NO: 14 - DNA, 150 bp, Human papilloma virus
gaatgaaacagatacagatgtagatggatttatagacaatacacttataaacaatacacaggaagacagggagacagctcaacaattattgcaagtacaaacagcacatgcagatgcacagacgttgcaaaaactaaaacgaaagtatat

SEQ ID NO: 15 - DNA, 150 bp, Human papilloma virus
aacagatacaggttcagacatggtagattttattgatgattctacacatatttgtatacaggcagagcgtgagacagcacaggtacttttgaatatgcaagaggcccaaagggatgcacaaagggtgcgtgccctaaaacgaaagtatac

SEQ ID NO: 16 - DNA, 146 bp, Human papilloma virus
aagcaccgaatctgatttggatgggtttatagacaatagtaatataatatctacacaggcagaaagggagacagctcagcagttgttacatgccaaaacacacatgcagatacacagacgctgcagaaattaaaacgaaagtattt

SEQ ID NO: 17 - DNA, 153 bp, Human papilloma virus
aggggatacagatgagtcggaaatgggggattttattgataatgcacatataccaaatatatatgcacaacaggaaattgcacaggcattgtatcagtcacagcaagcaaatgcagacaatgaggctatacgtgttctaaaacgaaagtttac

SEQ ID NO: 18 - DNA, 144 bp, Human papilloma virus
ggtggaggacagtgggtatgacatggtggactttattgatgacagcaatattacacacaattcactggaagcacaggcattgtttaacaggcaggaggcggacacccattatgcgactgtgcaggacctaaaacgaaagtattt

SEQ ID NO: 19 - DNA, 144 bp, Human papilloma virus; identical to SEQ ID NO: 18

SEQ ID NO: 20 - DNA, 144 bp, Human papilloma virus
agtggaggatagtgggttggatatggtggactttatagatgacaggcctattacacacaattccatggaagcacaggcattgttaaacgagcaggaggcggatgctcattatgcggctgtgcaggacctaaaacgaaagtattt

SEQ ID NO: 21 - DNA, 150 bp, Human papilloma virus
agaggatagtggatttgatatgatagattttattgataatagtgttgtggcagaggaacatgtagaactaagtaatgcacaggcacttttacatgtacagcagacatgtgcagatgctgctgacctgtgcgagttaaaacgaaagtacat

SEQ ID NO: 22 - DNA, 150 bp, Human papilloma virus
agaggaagatagtgggtttgatatggtagattttattaataatacattagaagacagttgtacagaccacagcagtgcgcaggctctgttaaatgcacaacaagcggatgctgatgctgctatagtgcaggagttaaaacgaaagtacat

SEQ ID NO: 23 - DNA, 147 bp, Human papilloma virus
tgtagacgatagtgggttagatcttgtggattttgtagataatagtacagtaatacatacaaagcaggtacatgcacaagccttattaaataaacaacaagcacatgcagatcaggaggcagtacaggcactaaaacgaaagctatt

SEQ ID NO: 24 - DNA, 144 bp, Human papilloma virus
ggaggacagtgggcttgatatggtggactttattgataatagtgtgtcacaggtagaggggcaggaaaatccacaggcattgttacatgcccaacagctgcaggcagatgtagaggcagtgcaacaattaaaacgaaagtatat

SEQ ID NO: 25 - DNA, 141 bp, Human papilloma virus
cattgacacaggggaagacctagtagacttcatagatacaaggcgccccggggatgggcaggaagtgccgcttgcgttgttcgttcaacaaaatgcacaggatgacgctgcaacggtgcaggcactaaaacgaaagtatac

SEQ ID NO: 26 - DNA, 150 bp, Human papilloma virus
aacagatacaggttcagacttggcagactttattgatgatactacagatatttgtgtacaggcagagcgcgagacagcacaggtactgtataatatgcaagaggcccaaagggatgcacaatcagtgcgtgccttaaaacgaaagtatgg

SEQ ID NO: 27 - DNA, 141 bp, Human papilloma virus
tgaggacaggggagaagatctggtagactttatagacacaagatccttaggggatgggcaggaagtgccgttagatttgttcgtgcaacaaaatgcacgggatgacgctgcaaccgtgcaggccctaaaacgaaagtatac

SEQ ID NO: 28 - DNA, 141 bp, Human papilloma virus
tgcagatacaggagaggatctagtagatttcatagatacacgatatccaggggatgggcaggaagtgccgttagaactgtttgttcaacaaaatgcacaggatgacgctgcagcggtgcatgcactaaaacgaaagtatat

SEQ ID NO: 29 - DNA, 20 bp, Artificial Sequence
  Description of Artificial Sequence Synthetic primer
caggcagaattagagrcagc

SEQ ID NO: 30 - DNA, 22 bp, Artificial Sequence
  Description of Artificial Sequence Synthetic primer
tccaccacawactttcgtttta

SEQ ID NO: 31 - DNA, 20 bp, Artificial Sequence
  Description of Artificial Sequence Synthetic primer
caggcagaattagagrcagc

SEQ ID NO: 32 - DNA, 149 bp, Human papilloma virus
cagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtatt

SEQ ID NO: 33 - DNA, 149 bp, Human papilloma virus
aacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttg

SEQ ID NO: 34 - DNA, 149 bp, Human papilloma virus
tagtgatactggggaggatatggttgactttattgacaattgtaatgtatacaacaatcaggcagaagcagagacagcacaggcattgtttcatgcacaggaagcggaggaacatgcagaggctgtgcaggttctaaaacgaaagtatg

SEQ ID NO: 35 - DNA, 149 bp, Human papilloma virus
agatgacagtggcacggatttactagagtttatagatgattctatggaaaatagtatacaggcagacacagaggcagcccgggcattgtttaatatacaggaaggggaggatgatttaaatgctgtgtgtgcactaaaacgaaagtttg

SEQ ID NO: 36 - DNA, 149 bp, Human papilloma virus
ctgtgacaggggggaggatatggtggactttataaatgatacagatatattaaacatacaggcagaaacagagacagcacaagcattatttcatgcacaggaggagcaaacacacaaagaggctgtacaggtcctaaaacgaaagtatg

SEQ ID NO: 37 - DNA, 149 bp, Human papilloma virus
aacagatacaggttcagacctggcagactttattgatgattccacagatatttgtgtacaggcagagcgtgagacagcacaggtacttttacatatgcaagaggcccaaagggatgcacaagcagtgcgtgccttaaaacgaaagtata

SEQ ID NO: 38 - DNA, 149 bp, Human papilloma virus
aacagatacagggtcggatatggtagattttattgacacacaattatccatttgtgaacaggcagagcaagagacagcacaggcattgttccatgcgcaggaagttcagaatgatgcacaggtgttgcatcttttaaaacgaaagtttg

SEQ ID NO: 39 - DNA, 149 bp, Human papilloma virus
agatgatacaggatctgatttaataaactttatagatagtgaaactagtatttgcagtcaggcggaacaggagacagcacgggcgttgtttcaggcccaagaattacaggcaaacaaagaggctgtgcatcagttaaaacgaaagtttc

SEQ ID NO: 40 - DNA, 149 bp, Human papilloma virus
atatgatagtggaacagatctaatagattttatagatgattcaaatataaataatgaacaggcagaacatgaggcagcccgggcattgtttaatgcacaggaaggggaggatgatttacatgctgtgtctgcagtaaaacgaaagttta

SEQ ID NO: 41 - DNA, 148 bp, Human papilloma virus
ggatgaaatagatacagatttagatggatttatagacgattcatatatacaaaatatacaggcagacgcagaaacagtcaacaattgttgcaagtacaaacagcacatgcagataaacagacgttgcaaaaactaaaacgaaagtata

SEQ ID NO: 42 - DNA, 149 bp, Human papilloma virus
agacgatagtggtacagatttaatagagtttatagatgattcagtacaaagtactacacaggcagaagcagaggcagcccgagcgttgtttaatgtacaggaaggggtggacgatataaatgctgtgtgtgcactaaaacgaaagtttg

SEQ ID NO: 43 - DNA, 149 bp, Human papilloma virus; identical to SEQ ID NO: 12

SEQ ID NO: 44 - DNA, 149 bp, Human papilloma virus
aaatgatacagggtctgatataatagactttatagatacaaataacagtatttgcagtcaggcggaacaagagacagcacgggcgttgtttcaggtccaagaaacacaggcacacaaagaggctgcacagcatctaaaacgaaagtttt

SEQ ID NO: 45 - DNA, 142 bp, Human papilloma virus
ggtggaggacagtgggtatgacatggtggactttattgatgacagcaatattacacacaattcactggaagcacaggcattgtttaacaggcaggaggcggacacccattatgcgactgtgcaggacctaaaacgaaagtat

SEQ ID NO: 46 - DNA, 142 bp, Human papilloma virus
ggtggaggacagtgggtatgacatggtggactttattgatgacaggcatattacacaaaattctgtggaagcacaggcattgtttaataggcaggaggcggatgctcattatgcgactgtgcaggacctaaaacgaaagtat

SEQ ID NO: 47 - DNA, 33 bp, Artificial Sequence
  Description of Artificial Sequence Synthetic primer
caactccatctacacaggcagaaacagagacag

SEQ ID NO: 48 - DNA, 33 bp, Artificial Sequence
  Description of Artificial Sequence Synthetic primer
caactccatctgaacaggcagagctagagacag

SEQ ID NO: 49 - DNA, 33 bp, Artificial Sequence
  Description of Artificial Sequence Synthetic primer
caactccatctaatcaggcagaagcagagacag

SEQ ID NO: 50 - DNA, 33 bp, Artificial Sequence
  Description of Artificial Sequence Synthetic primer
caactccatctatacaggcagacacagaggcag

SEQ ID NO: 51 - DNA, 33 bp, Artificial Sequence
  Description of Artificial Sequence Synthetic primer
caactccatctatacaggcagaaacagagacag

SEQ ID NO: 52 - DNA, 33 bp, Artificial Sequence
  Description of Artificial Sequence Synthetic primer
caactccatctgtacaggcagagcgtgagacag

SEQ ID NO: 53 - DNA, 33 bp, Artificial Sequence
  Description of Artificial Sequence Synthetic primer
caactccatctgaacaggcagagcaagagacag

SEQ ID NO: 54 - DNA, 33 bp, Artificial Sequence
  Description of Artificial Sequence Synthetic primer
caactccatctgaacaggcagaacatgaggcag

SEQ ID NO: 55 - DNA, 33 bp, Artificial Sequence
  Description of Artificial Sequence Synthetic primer
caactccatctatacaggcagacgcagaaacag

SEQ ID NO: 56 - DNA, 33 bp, Artificial Sequence
  Description of Artificial Sequence Synthetic primer
caactccatctacacaggcagaagcagaggcag

SEQ ID NO: 57 - DNA, 33 bp, Artificial Sequence
  Description of Artificial Sequence Synthetic primer
caactccatctgtacaggcagagcgcgagacag

SEQ ID NO: 58 - DNA, 33 bp, Artificial Sequence
  Description of Artificial Sequence Synthetic primer
caactccatctagtcaggcggaacaagagacag

SEQ ID NO: 59 - DNA, 27 bp, Artificial Sequence
  Description of Artificial Sequence Synthetic primer
tgcttcctgtgcagtaaacaacgcatg

SEQ ID NO: 60 - DNA, 24 bp, Artificial Sequence
  Description of Artificial Sequence Synthetic primer
gacctcctgcgcatggaacaatgc

SEQ ID NO: 61 - DNA, 23 bp, Artificial Sequence
  Description of Artificial Sequence Synthetic primer
cgcttcctgtgcatgaaacaatg

SEQ ID NO: 62 - DNA, 25 bp, Artificial Sequence
  Description of Artificial Sequence Synthetic primer
cccttcctgtatattaaacaatgcc

SEQ ID NO: 63 - DNA, 27 bp, Artificial Sequence
  Description of Artificial Sequence Synthetic primer
ctcctcctgtgcatgaaataatgcttg

SEQ ID NO: 64 - DNA, 24 bp, Artificial Sequence
  Description of Artificial Sequence Synthetic primer
ggcctcttgcatatgtaaaagtac

SEQ ID NO: 65 - DNA, 23 bp, Artificial Sequence
  Description of Artificial Sequence Synthetic primer
aacttcctgcgcatggaacaatg

SEQ ID NO: 66 - DNA, 23 bp, Artificial Sequence
  Description of Artificial Sequence Synthetic primer
taattcttgggcctgaaacaacg

SEQ ID NO: 67 - DNA, 23 bp, Artificial Sequence
  Description of Artificial Sequence Synthetic primer
cccttcctgtgcattaaacaatg

SEQ ID NO: 68 - DNA, 25 bp, Artificial Sequence
  Description of Artificial Sequence Synthetic primer
gtgctgtttgtacttgcaacaattg

SEQ ID NO: 69 - DNA, 23 bp, Artificial Sequence
  Description of Artificial Sequence Synthetic primer
cccttcctgtacattaaacaacg

SEQ ID NO: 70 - DNA, 23 bp, Artificial Sequence
  Description of Artificial Sequence Synthetic primer
ggcttcctgcacattaaacaagg

SEQ ID NO: 71 - DNA, 23 bp, Artificial Sequence
  Description of Artificial Sequence Synthetic primer
tgtttcttggacctgaaacaacg

SEQ ID NO: 72 - DNA, 19 bp, Artificial Sequence
  Description of Artificial Sequence Synthetic primer
caggcagaaacagagacag

SEQ ID NO: 73 - DNA, 19 bp, Artificial Sequence
  Description of Artificial Sequence Synthetic primer
caggcagagctagagacag

SEQ ID NO: 74 - DNA, 19 bp, Artificial Sequence
  Description of Artificial Sequence Synthetic primer
caggcagaagcagagacag

SEQ ID NO: 75 - DNA, 19 bp, Artificial Sequence
  Description of Artificial Sequence Synthetic primer
caggcagacacagaggcag

SEQ ID NO: 76 - DNA, 19 bp, Artificial Sequence
  Description of Artificial Sequence Synthetic primer
caggcagagcgtgagacag

SEQ ID NO: 77 - DNA, 19 bp, Artificial Sequence
  Description of Artificial Sequence Synthetic primer
caggcagagcaagagacag

SEQ ID NO: 78 - DNA, 19 bp, Artificial Sequence
  Description of Artificial Sequence Synthetic primer
caggcggaacaggagacag

SEQ ID NO: 79 - DNA, 19 bp, Artificial Sequence
  Description of Artificial Sequence Synthetic primer
caggcagaacatgaggcag

SEQ ID NO: 80 - DNA, 19 bp, Artificial Sequence
  Description of Artificial Sequence Synthetic primer
caggcagacgcagaaacag

SEQ ID NO: 81 - DNA, 19 bp, Artificial Sequence
  Description of Artificial Sequence Synthetic primer
caggcagaagcagaggcag

SEQ ID NO: 82 - DNA, 19 bp, Artificial Sequence
  Description of Artificial Sequence Synthetic primer
caggcagagcgcgagacag

SEQ ID NO: 83 - DNA, 19 bp, Artificial Sequence
  Description of Artificial Sequence Synthetic primer
caggcggaacaagagacag

SEQ ID NO: 84 - DNA, 23 bp, Artificial Sequence
  Description of Artificial Sequence Synthetic primer
tgcttcctgtgcagtaaacaacg

SEQ ID NO: 85 - DNA, 23 bp, Artificial Sequence
  Description of Artificial Sequence Synthetic primer
gacctcctgcgcatggaacaatg

SEQ ID NO: 86 - DNA, 23 bp, Artificial Sequence
  Description of Artificial Sequence Synthetic primer
cccttcctgtatattaaacaatg

SEQ ID NO: 87 - DNA, 23 bp, Artificial Sequence
  Description of Artificial Sequence Synthetic primer
ctcctcctgtgcatgaaataatg

SEQ ID NO: 88 - DNA, 24 bp, Artificial Sequence
  Description of Artificial Sequence Synthetic primer
tgctgtttgtacttgcaacaattg

SEQ ID NO: 89 - DNA, 20 bp, Artificial Sequence
  Description of Artificial Sequence Synthetic primer
cgtccmarrggawactgatc

SEQ ID NO: 90 - DNA, 20 bp, Artificial Sequence
  Description of Artificial Sequence Synthetic primer
gcmcagggwcataayaatgg

(19) United States
(12) Patent Application Publication
(10) Pub. No.: US 2015/0376725 A1
(43) Pub. Date: Dec. 31, 2015

(54) HPV Detection in Urine

(71) Applicant: Trovagene, Inc., San Diego, CA (US)

(72) Inventor: Cecile Rose Vibat, San Diego, CA (US)

(21) Appl. No.: 14/018,377

(22) Filed: Sep. 4, 2013

(51) Int. Cl. C12Q 1/70

(52) U.S. Cl. CPC C12Q 1/708; C12Q 2600/158

(57) ABSTRACT

The disclosure provides compositions and methods for the detection of human papillomavirus (HPV) nucleic acids in a urine sample from a human subject. The nucleic acids may be from one or more high risk forms of HPV. The urine sample is minimally processed, without fractionation into cell-free and cell-containing portions, before preparation of the nucleic acids for detection of HPV sequences.

### FIELD OF THE DISCLOSURE

This disclosure relates to the fields of medicine and clinical diagnostics based on molecular techniques. The techniques include methods for detecting human papillomavirus (HPV) nucleic acid molecules in urine samples from human subjects as an indicator of HPV infection in the subject. The detection methods include type-specific detection of HPV infections and the detection of HPV genotypes.

### BACKGROUND OF THE DISCLOSURE

As recently as 2011, the detection of human papillomavirus (HPV) infections by use of a urine sample included a concern over the presence of soluble PCR inhibitors in urine (see page 1745, right column, in Bissett et al.). This concern led Bissett et al. to test three different protocols for the processing of urine samples as part of extraction of HPV nucleic acids. One protocol, which was reported as having the better sensitivity among the three, was centrifugation of urine (1 ml) at 13,000 rpm for 20 minutes, removal of the resulting supernatant, resuspension in 300 μl before freezing at −25° C. until use for nucleic acid extraction. The other two protocols were storing unprocessed urine at −25° C. with i) centrifugation and resuspension after defrosting (and immediately before use for nucleic acid extraction); and ii) no processing after defrosting (and direct use for nucleic acid extraction).

Bissett et al.'s report of superior sensitivity in the cellular pellet over unfractionated urine is consistent with the use of cellular pellets by multiple investigators as reviewed by Vorsters et al. in 2012 and Enerly et al. in 2013. Two exceptions noted by Vorsters et al. are Strauss et al., who reported the fractionation of urine by centrifugation into supernatant and a cell pellet resuspended in a smaller volume of supernatant, and Smits et al., who reported the extraction of male urine with guanidinium thiocianate lysis and isolation as described by Boom et al. The use of dilution to reduce or eliminate the effects of PCR inhibitors was explicitly noted by Strauss et al. (see page 538). Strauss et al. also reported a peak sensitivity of 58.4% in the fraction formed by concentration of a cell pellet by resuspension with urine supernatant (see page 540).

The citation of documents herein is not to be construed as reflecting an admission that any is relevant prior art. Moreover, their citation is not an indication of a search for relevant disclosures. All statements regarding the date(s) or contents of the documents is based on available information and is not an admission as to their accuracy or correctness.

### BRIEF SUMMARY OF THE DISCLOSURE

The disclosure relates to methods for the detection of human papillomavirus (HPV) nucleic acid molecules in urine samples from human subjects. The disclosed methods do not fractionate urine into cell-free and cell-containing fractions, or soluble and insoluble fractions, and surprisingly provide sensitivities that are higher than those reported for cellular pellets. The lack of fractionation allows the preparation of nucleic acids from urine without the loss of molecules due to separation techniques. The surprising sensitivities from unfractionated urine was observed even in cases of a frozen sample that is defrosted before preparation and detection of HPV nucleic acids.

In a first aspect, the disclosure includes a method of detecting human papillomavirus (HPV) infection by use of a urine sample from a human subject. The sample may be from a female or male subject, with the detection optionally tailored to the type(s) of HPV infection observed or known to a skilled person for a female subject or a male subject.

The disclosed methods include the isolation, or preparation, of nucleic acids from a urine sample collected from a human subject. The urine sample is unfractionated prior to the isolation, or preparation. Therefore, there is no processing of a urine sample into cell-containing and cell-free fractions, or soluble and insoluble fractions. And the urine sample contains the cell-free HPV nucleic acids, HPV virions or viral particles, and cell associated HPV nucleic acids as present in a human subject's urine.

In some embodiments, the urine sample is treated to reduce nucleic acid degradation. The treatment may be after collection and before the sample is further handled, manipulated, or stored. In other embodiments, the treatment may be after collection and further handling, manipulation, or storage but before extraction, isolation, or preparation of nucleic acid molecules in the sample. Non-limiting examples of treatment include freezing the sample, lowering the temperature of the sample, heat inactivation of nucleases in the sample, increasing pH, addition of an agent to reduce degradation, or a combination of these techniques as known to the skilled person. In some cases, the added agent increases pH, increases the salt concentration or ionic strength, or is ethylenediaminetetraacetic acid (EDTA), guanidine-HCl guanidine isothiocyanate (GITC) or other chaotropic salt, N-lauroylsarcosine, and sodium dodecylsulphate. Any treatment as described herein results in a treated urine sample, from which nucleic acids may be prepared as described below.

The isolation, or preparation, of HPV nucleic acids is performed in the presence of an exogenous or added nucleic acid carrier agent. The carrier agent may be any known to the skilled person for improving the isolation of nucleic acids without interfering with subsequent isolation steps. Non-limiting examples include carrier RNA, carrier DNA, linear polyacrylamide (LPA), and glycogen.

The isolated HPV nucleic acids may be detected by means known to the skilled person. In some embodiments, the detection may include the amplification of one or more HPV sequences. In some cases, the amplification may include the polymerase chain reaction (PCR), using forward and reverse primers. One or both of the primers may include non-HPV sequences and/or a detectable label. In other embodiments, the detection may comprise amplification followed by sequencing of the amplified molecules. In further embodiments, the detection may comprise direct sequencing of nucleic acids without prior amplification. In additional alternative embodiments, the detection includes a technique selected from nucleic acid hybridization; Cyclic Probe Reaction; Single-Strand Conformation Polymorphism (SSCP); Strand Displacement Amplification (STA); and Restriction Fragment Length Polymorphism (RFLP) as non-limiting examples.

The detection of HPV nucleic acid sequence(s) in a urine sample indicates the presence of HPV nucleic acid molecules in the urine source of the sample. The nucleic acid molecules may be in the form of cell-free nucleic acids, HPV virions or HPV viral particles, cell associated nucleic acids, such as HPV nucleic acid molecules within cells or associated with the external surface of a cell, or any combination of the above. One non-limiting example of HPV nucleic acid molecules within a cell is one or more episomal copies of HPV genetic material within a cell. Additionally, the detection of HPV nucleic acid molecules in a urine sample indicates the presence of an HPV infection in the subject. In some embodiments, the infection may be of cells in the urinary tract. In other embodiments, the infection may be of cells from outside the urinary tract, such as cervical cells as a non-limiting example.

The detected HPV may be of a single type, such as HPV 16 or 18 as non-limiting examples. Alternatively, the detection may be of two or more types, such as HPV 16 and 18, or HPV 16, 18, 31, 33, 35, 39, 45, 51, 52, 56, 58, 59, and 68. In some cases, the detection may be of at least one “high risk” HPV type and at least one “low risk” HPV type. Therefore, the detection includes the detection of any one or more HPV types.

In some embodiments, a method may include (a) isolating HPV nucleic acids, from an unfractionated urine sample collected from a human subject, from the sample and in the presence of an exogenous or added nucleic acid carrier agent, and (b) detecting the isolated HPV nucleic acids. In some cases, the detected presence of HPV nucleic acids may be used to indicate the presence of an HPV infection in the subject. The isolation may include the removal of non-nucleic acid or non-DNA cellular components, such as proteins and lipids that are not associated with nucleic acids.

In a second aspect, the disclosure includes a method of preparing HPV nucleic acids present in a urine sample from a human subject. The disclosed methods may include isolating, or preparing nucleic acids from an unfractionated urine sample collected from a human subject as described herein. The resulting nucleic acids may be assayed, tested, or used in any suitable manner known to the skilled person. The resulting nucleic acids are cell-free HPV nucleic acids, are from HPV virions or viral particles, or from cell associated HPV nucleic acids as present in a human subject's urine. The urine sample is optionally treated to reduce nucleic acid degradation as described herein. Additionally, the isolation, or preparation, of HPV nucleic acids is performed in the presence of an exogenous or added nucleic acid carrier agent as described herein.

In some embodiments, a method may include isolating, from an unfractionated urine sample collected from a human subject, HPV nucleic acids in the sample and in the presence of an exogenous or added nucleic acid carrier agent. The isolation may include the removal of non-nucleic acid or non-DNA cellular components, such as proteins and lipids as non-limiting examples (and including those that are not associated with nucleic acids. The resulting isolated material is a composition that includes both HPV nucleic acid molecules and non-HPV nucleic acid molecules, such as those from the subject's cellular genome. The isolated HPV nucleic acids may be of a single type, such as HPV 16 or 18 as non-limiting examples. Alternatively, the nucleic acids may be of two or more types, such as HPV 16 and 18, or HPV 16, 18, 31, 33, 35, 39, 45, 51, 52, 56, 58, 59, and 68. In some cases, the nucleic acids may be of at least one “high risk” HPV type and at least one “low risk” HPV type.

In an additional aspect, the disclosure includes a composition containing HPV nucleic acids isolated or prepared by a disclosed method. The composition contains reduced amounts of proteins and other biological or cellular components originally present with the HPV nucleic acids in a urine sample. The reduction of the proteins and other biological or cellular components may be by any method described herein. The composition may contain the nucleic acids in combination with a suitable buffering agent and/or chelating agent.

The disclosure further provides a diagnostic kit for detecting HPV, comprising: reagents to perform all or part of the methods disclosed herein.

### BRIEF DESCRIPTION OF THE FIGURES

FIG. 1 shows representative results from capillary electrophoresis analysis of urine samples from human subjects infected with HPV-16 (top) and HPV-54 (bottom). The indicated basepairs (0, 20, 40, 60, 80, 100, and 120) reflects the lengths of detected nucleic acid molecules from a PCR reaction using a set of high risk (HR) primers as disclosed herein. The sample trace is in blue, with a molecular weight marker in orange. The Y-axis scale indicates relative fluorescence units from 0 to 10,000.

FIG. 2 shows representative results from capillary electrophoresis analysis of urine samples from an uninfected (negative control) human subject and from human subjects infected with HPV-16, HPV-18, and HPV-81, respectively. The indicated basepairs (0, 20, 40, 60, 80, 100, and 120) reflects the lengths of detected HPV molecules amplified by PCR using the same high risk (HR) primers as in FIG. 1. The shaded area indicates the 93-96 bp range. The Y-axis scale indicates relative fluorescence units from 0 to 10,000.

### DETAILED DESCRIPTION OF MODES OF PRACTICING THE DISCLOSURE

General

Human papillomaviruses (HPVs) are epitheliotropic viruses associated with benign and malignant lesions of cutaneous and mucosal epithelia. There is well documented causative connection between HPV infection and subsequent development of cervical cancer. There are also observations associating HPV infection with cancers of the head and neck, respiratory tissue and breast. (Braakhuis et al., 2004, J. Natl. Cancer Inst. 96(13): 998-1006; Dahlstrand et al., 2004, Anticancer Res. 24(3b): 1829-35; Daling et al., 2004, Cancer 101 (2): 270-80; Ha et al., 2004, Crit. Rev. Oral Biol. Med. 15(4): 188-96; Hafkamp et al., Acta Otolaryngol. 124(4): 520-6; Harwood et al., 2004, Br. J. Dermatol. 150(5):949-57; Rees et al., 2004, Clin. Otolaryngol. 29(4):301-6; Widschwendter et al., 2004, J. Clin. Virol. 31(4):292-7). More than 100 different types of HPV have been identified to date (Antonsson, A., et al., 2000, J. Virol. 74:11636-11641; Chan, S. Y., et al. 1995, J. Virol. 69:3074-3083; de Villiers, E. M., et al. 2004, Virology 324:17-27), of which 40 have been reported in anogenital infections (de Villiers E-M. 2001, Papillomavirus Rep. 12:57-63; Villiers E M et al., 2004, Virology. June 20; 324(1):17-27).

It is accepted that nearly 100% of invasive cervical cancers and high-grade precancerous intraepithelial neoplasias are associated with infection by high-risk HPV infection.

The disclosure provides methods and compositions for the preparation and detection of HPV nucleic acids in a urine sample provided by a human subject. The disclosure provides reagents and methods for the preparation of HPV nucleic acids from an unfractionated, or a non-concentrated, urine sample. Additionally, the disclosure provides primers and probes that recognize and bind HPV nucleic acid molecules for detecting them among nucleic acids present in a human urine sample. In some embodiments, the primers and probes detect sequences within the E1 gene of HPV. In some cases, the detection is of high risk forms of HPV to identify female subjects at risk of developing, or in the early stages of, cervical carcinoma. In other cases, the detection is of HPV infection in male subjects.

To facilitate understanding of the disclosure, a number of terms are defined below.

The terms “detect” and “analyze” HPV in relation to a nucleic acid sequence, refer to the use of any method of observing, ascertaining, measuring, or quantifying signals indicating the presence of the target nucleic acid sequence in a sample or the absolute or relative quantity of that target nucleic acid sequence in a sample. Methods can be combined with nucleic acid labeling methods to provide a signal by, for example: fluorescence, radioactivity, colorimetry, gravimetry, X-ray diffraction or adsorption, magnetism, enzymatic activity and the like. The signal can then be detected and/or quantified, by methods appropriate to the type of signal, to determine the presence or absence, of the specific DNA sequence of interest.

To “quantify” in relation to a nucleic acid sequence, refers to the use of any method to study the amount of a particular nucleic acid sequence, including, without limitation, methods to determine the number of copies of a nucleic acid sequence or to determine the change in quantity of copies of the nucleic acid sequence over time, or to determine the relative concentration of a sequence when compared to another sequence.

To assist in detection and analysis, specific DNA sequences can be “amplified” in a number of ways, including, but not limited to cycling probe reaction (Bekkaoui, F. et al, BioTechniques 20, 240-248 (1996), polymerase chain reaction (PCR), nested PCR, PCR-SSCP (single strand conformation polymorphism), ligase chain reaction (LCR) (F. Barany Proc. Natl. Acad. Sci USA 88:189-93 (1991)), cloning, strand displacement amplification (SDA) (G. K. Terrance Walker et al., Nucleic Acids Res. 22:2670-77 (1994), and variations such as allele-specific amplification (ASA).

The term “gene” refers to a DNA sequence that comprises control and coding sequences necessary for the transcription of an RNA sequence. The term HPV “genome” refers to the complete gene complement of HPV.

The terms “oligonucleotide” and “polynucleotide” and “polymeric” nucleic acid are interchangeable and are defined as a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide can be generated in any manner, including chemical synthesis, DNA replication, reverse transcription, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also can be said to have 5′ and 3′ ends.

When two different, non-overlapping oligonucleotides anneal to different regions of the same linear complementary nucleic acid sequence, and the 3′ end of one oligonucleotide points towards the 5′ end of the other, the former can be called the “upstream” oligonucleotide and the latter the “downstream” oligonucleotide.

The term “primer” refers to an oligonucleotide which is capable of acting as a point of initiation of synthesis when placed under conditions in which primer extension is initiated. An oligonucleotide “primer” can occur naturally, as in a purified restriction digest or be produced synthetically. Amplification of a nucleic acid molecule includes multiple, repeated instances of primer extension.

A primer is selected to be “substantially” complementary to a strand of specific sequence of the template. A primer must be sufficiently complementary to hybridize with a template strand for primer elongation to occur. A primer sequence need not reflect the exact sequence of the template. For example, a non-complementary nucleotide fragment can be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template primer complex for synthesis of the extension product of the primer.

A “target” nucleic acid is a nucleic acid sequence to be evaluated by hybridization, amplification or any other means of analyzing a nucleic acid sequence, including a combination of analysis methods.

“Hybridization” methods involve the annealing of a complementary sequence to the target nucleic acid (the sequence to be analyzed). The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modern biology. Hybridization encompasses, but is not limited to, slot, dot and blot hybridization techniques. Hybridization may involve partial complementarity or complete complementarity. Hybridization, regardless of the method used, requires some degree of complementarity between the sequence being analyzed (the target sequence) and the oligonucleotide used to perform the test (the probe). The term “hybridization” as used herein includes “any process by which a strand of nucleic acid joins with a complementary strand through base pairing” (Coombs, Dictionary of Biotechnology, Stockton Press, New York N.Y. (1994).

The complement of a nucleic acid sequence as used herein refers to an oligonucleotide which, when aligned with the nucleic acid sequence such that the 5′ end of one sequence is paired with the 3′ end of the other, is in “antiparallel association.” Specific bases not commonly found in natural nucleic acids can be included in the nucleic acids of the present disclosure and include, for example, inosine and 7-deazaguanine. Complementarity need not be perfect; stable duplexes can contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs, and relative position of any mismatched base pair(s) in the oligonucleotide and in relation to other mismatched and complementary base pairs.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value can be calculated by the equation: Tₘ=81.5+0.41(% G+C), when a nucleic acid is in aqueous solution at 1 M NaCl (see e.g., Anderson and Young, Quantitative Filter Hybridisation, in Nucleic Acid Hybridisation (1985). Other references include more sophisticated computations which take structural as well as sequence characteristics into account for the calculation of Tₘ.

The term “probe” as used herein refers to an oligonucleotide (i.e., a sequence of nucleotides), whether occurring naturally as in a purified restriction digest or produced synthetically, which forms a duplex structure or other complex with a sequence in another nucleic acid, due to complementarity or other means of reproducible attractive interaction, of at least one sequence in the probe with a sequence in the other nucleic acid. Probes are useful in the detection, identification and isolation of particular gene sequences. The disclosure includes a probe that is labeled with any “reporter molecule,” so that it is detectable in any detection system, including, but not limited to, enzyme (e.g., ELISA, as well as enzyme-based histochemical assays), fluorescent, radioactive, and luminescent systems. In some cases, an oligonucleotide of interest (i.e., to be detected) will be labeled with a reporter molecule. In other cases, both the probe and oligonucleotide of interest will be labeled. The present disclosure is not limited to any particular detection system or label.

The term “label” as used herein refers to any atom or molecule which can be used to provide a detectable (preferably quantifiable) signal, and which can be attached to a nucleic acid or protein. Labels provide signals detectable by any number of methods, including, but not limited to, fluorescence, radioactivity, colorimetry, gravimetry, X-ray diffraction or absorption, magnetism, and enzymatic activity.

The term “substantially single-stranded” when used in reference to a nucleic acid target means that the target molecule exists primarily as a single strand of nucleic acid in contrast to a double-stranded target which exists as two strands of nucleic acid which are held together by inter-strand base pairing interactions.

“Oligonucleotide primers matching or complementary to a gene sequence” refers to oligonucleotide primers capable of facilitating the template-dependent synthesis of single or double-stranded nucleic acids. Oligonucleotide primers matching or complementary to a gene sequence can be used in PCRs, RT-PCRs and the like.

“Nucleic acid sequence” as used herein refers to an oligonucleotide, nucleotide or polynucleotide, and fragments or portions thereof, and to DNA or RNA of genomic or synthetic origin which can be single- or double-stranded, and represent the sense or antisense strand.

“Amplification” is defined as the production of additional copies of a nucleic acid sequence and is generally carried out using polymerase chain reaction or other technologies well known in the art (e.g., Dieffenbach and Dveksler, PCR Primer, a Laboratory Manual, Cold Spring Harbor Press, Plainview, N.Y. [1995]). As used herein, the term “polymerase chain reaction” (“PCR”) refers to the method of K. B. Mullis (U.S. Pat. Nos. 4,683,195 and 4,683,202, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

As used herein, the term “polymerase” refers to any enzyme suitable for use in the amplification of nucleic acids of interest. It is intended that the term encompass such DNA polymerases as Taq DNA polymerase obtained from Thermus aquaticus, although other polymerases, both thermostable and thermolabile are also encompassed by this definition. With PCR, it is possible to amplify a single copy of a specific target sequence in genomic DNA to a level that can be detected by several different methodologies (e.g., staining, hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the terms “PCR product” and “amplification product” refer to the resultant mixture of compounds after two or more cycles of the PCR steps of denaturation, annealing and extension are complete. These terms encompass the case where there has been amplification of one or more segments of one or more target sequences.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity can be “partial,” in which only some of the nucleic acids' bases are matched according to the base pairing rules. Or, there can be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There can be partial homology or complete homology (i.e., identity). A partially complementary sequence is one that at least partially inhibits a completely complementary sequence from hybridizing to a target nucleic acid is referred to using the functional term “substantially homologous.”

As used herein the term “hybridization complex” refers to a complex formed between two nucleic acid sequences by virtue of the formation of hydrogen bonds between complementary G and C bases and between complementary A and T bases; these hydrogen bonds can be further stabilized by base stacking interactions. The two complementary nucleic acid sequences hydrogen bond in an antiparallel configuration. A hybridization complex can be formed in solution (e.g., COt or ROt analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in situ hybridization, including FISH (fluorescent in situ hybridization)).

The term “sample” as used herein is used in its broadest sense. A biological sample suspected of containing nucleic acid can comprise, but is not limited to, genomic DNA (in solution or bound to a cell or cellular components), cDNA (in solution or bound to a solid support), and the like.

The terms “urinary tract” and “genitourinary tract” as used herein refer to the organs and ducts which participate in the secretion and elimination of urine from the body.

The terms “transrenal DNA” and “transrenal nucleic acid” as used herein refer to nucleic acids that have crossed the kidney barrier. Transrenal DNA as used herein differs from miRNA. Specifically, transrenal DNA comprises randomness in the 3′ and 5′ ends, which is not present in miRNA.

Urine Sampling and Nucleic Acid Preparation for Analysis

As described herein, the disclosure provides a method that includes (a) isolating HPV nucleic acids, from an unfractionated urine sample collected from a human subject, in the sample and in the presence of an exogenous or added nucleic acid carrier agent, and (b) detecting the isolated HPV nucleic acids. In some embodiments, the detected presence of HPV nucleic acids indicates the presence of HPV in the subject. In some cases, the method may be used to detect the presence of an HPV infection in the subject. In additional embodiments, the presence of HPV nucleic acids may indicate a chronic or acute infection may be, or alternatively be the result of episomal HPV nucleic acids or nucleic acid integration into a cellular genome. The isolation may include the removal of non-nucleic acid or non-DNA cellular components, such as proteins and lipids that are not associated with nucleic acids.

The method may be used with a urine sample from a female or male subject. In the case of female urine, detection may be used to detect HPV infections observed or known to occur in a female subject. Non-limiting examples include infections of cervical tissues or cells, the vulva, and the linings of the vagina. In the case of male urine, the method may be used to detect HPV infections observed or known to occur in a male subject. Non-limiting examples include infection of tissues or cells of the penis. The disclosure also includes detection of HPV infections observed or known to occur in both female and male subjects, with infections of the genitourinary tract as a non-limiting example. The detection of HPV nucleic acid molecules in a urine sample indicates the presence of an HPV infection in the subject.

The disclosed methods are based in part upon the presence of HPV nucleic acid molecules in a urine sample obtained from a human subject. The nucleic acid molecules may be in the form of cell-free nucleic acids, HPV virions or HPV viral particles, cell associated nucleic acids, such as HPV nucleic acid molecules within cells or associated with the external surface of a cell, or any combination of the above. Additionally, nucleic acid molecules may be transrenal molecules that have crossed the kidney barrier from the blood into urine. Transrenal nucleic acids have a size distribution that is shorter in length than non-transrenal nucleic acids. While non-transrenal, cell-free and cell-associated nucleic acids released into urine from the genitourinary tract are expected to have a high molecular weight, transrenal nucleic acids that have crossed the kidney barrier from the bloodstream into urine are expected to be low molecular weight fragments. Low molecular weight transrenal NA sizes range from about 20 to 150. The disclosure includes methods for the detection of non-transrenal nucleic acids released into urine and/or transrenal nucleic acids.

In embodiments including transrenal HPV nucleic acids, the disclosed methods may be used to detect the presence of HPV infection in other body tissues or cells. As one non-limiting example, the methods may detect the presence of head and neck cancer, such as in the lip, mouth or oral cavity, nose and para-nasal sinuses, naso-pharynx, hypo-pharynx, larynx, throat, neck, or oropharynx, for example tonsils and the back (or base) of the tongue, as non-limiting examples. Therefore, embodiments of the disclosure include detection of tonsillar carcinomas, sino-nasal carcinomas, laryngeal squamous cell papillomas, and recurrent respiratory papillomatosis (RRP) as non-limiting HPV-induced tumors. In additional embodiments, the methods may be used to detect the presence of HPV-induced cancer of the anus and rectum.

The HPV nucleic acid molecules are detected by any known HPV sequence. In some embodiments, the HPV nucleic acid is from a “high-risk” (cancer-causing or cancer associated) type. In other embodiments, the HPV nucleic acid is from a “low-risk” (wart-causing) type. The distinction between these two groups is based on whether the HPV infection puts the human subject at risk for cancer. Non-limiting examples of high-risk (or “HR”) types are HPV 16, 18, 31, 33, 35, 39, 45, 51, 52, 56, 58, 59, and 68. Optionally, HPV type 66 may be considered HR based upon earlier scientific literature.

Before detection, the HPV nucleic acid molecules are isolated or prepared from a urine sample collected from a human subject. The urine sample is unfractionated prior to the isolation, or preparation. As used herein, the term fractionation refers to processing of a urine sample into two or more cell-containing and cell-free fractions, or two or more soluble and insoluble fractions. The term fractionate also includes the processing of a urine sample into a cell-containing fraction and a cell-free fraction that are subsequently recombined, even if the recombined material is concentrated or diluted relative to the original urine sample. The term does not, however, include the concentration of urine without fractionation of nucleic acids, such as by partial filtration or reducing liquid by butanol concentraction, of a urine sample to reduce its volume. Non-limiting examples of excluded methods for fractionating urine include centrifugation and filtering.

The unfractionated urine sample may be treated to reduce nucleic acid degradation in the sample. The treatment may be after collection and before the sample is further handled, manipulated, or stored. The addition of EDTA and/or a chaotropic salt is a particular technique that inhibits nucleic acid degradation and may be used in combination with each and every other aspect of the methods disclosed herein. In other embodiments, the treatment may be after collection and further handling, manipulation, or storage but before extraction, isolation, or preparation of nucleic acid molecules in the sample. Non-limiting examples of treatment include freezing the sample, lowering the temperature of the sample, heat inactivation of nucleases in the sample, increasing pH, addition of an agent to reduce degradation, or a combination of these techniques as known to the skilled person. In some cases, the added agent increases pH, increases the salt concentration or ionic strength, or is ethylenediaminetetraacetic acid (EDTA), guanidine-HCl guanidine isothiocyanate (GITC) or other chaotropic salt, N-lauroylsarcosine, and sodium dodecylsulphate. Any treatment as described herein results in a treated urine sample, from which nucleic acids may be isolated or prepared.

The disclosed treatment techniques address the possibility that DNA is subjected to DNases present in urine. An additional technique to address this possibility include obtaining urine samples when the urine has been held in the bladder for less than 12 hours. In some embodiments, the urine is held in the bladder for less than 5 hours, or for less than 2 hours before collection. In other embodiments, the urine is from the first void of the day after sleep.

As described herein, the isolating or preparing of HPV nucleic acids may include contacting a urine sample, optionally treated to reduce nucleic acid degradation, with a protease activity suitable for use in urine. The protease activity in the contacted sample may then be maintained or adjusted to proteolytic conditions. Non-limiting examples of adjusted to proteolytic conditions include additional of a buffering agent, and an increase in temperature. The skilled person is aware of multiple types of protease activities, and non-limiting examples include addition of proteinase K or other proteolytic enzymes. The selection of a protease activity that is compatible with the presence of EDTA and/or a chaotropic salt is also known to the skilled person.

The isolating, or preparing, of HPV nucleic acids is performed in the presence of an added (exogenous) nucleic acid carrier agent. The carrier agent is soluble, and may be any known to the skilled person for improving the isolation of nucleic acids without interfering with subsequent isolation steps as described herein. In some embodiments, the carrier agent is a polymer, such as a nucleic acid polymer as a non-limiting example. In some cases, the carrier agent is carrier RNA, tRNA, polyA RNA, carrier DNA, poly-dI, poly(dI-dC)•(dI-dC), poly(dGdC)•(dG-dC), poly(dA-dT)•(dA-dT), poly-dA, poly-dT, linear polyacrylamide (LPA), or glycogen. In some embodiments, the carrier, such as carrier RNA or tRNA, is added before the start of the protease treatment or after the start of treatment but before extraction of the HPV nucleic acids.

After proteolysis, the combination of HPV nucleic acids and added nucleic acid carrier agent is then precipitated or by adsorption of the nucleic acids to a solid adsorbent material. Methods for the precipitation of nucleic acids are known to the skilled person. Methods for the adsorption of nucleic acids to a solid adsorbent material, such as silica-based adsorbents, are also known and easily performed after addition of a chaotropic salt to the HPV nucleic acids. After adsorption, the bound nucleic acids may be washed (optionally with the same buffer used to apply the nucleic acids to the adsorbent material), and then eluted, such as with 10 mM Tris as a non-limiting example.

The isolated HPV nucleic acids may be detected by means known to the skilled person. In some embodiments, the detection is of a sequence within the E1, E2, E4, E6, E7, L1, and L2 regions of HPV. In some cases, the detection is of an HPV sequence represented by SEQ ID NOs: 1-28 (as shown in Table 1) or a fragment thereof. The detection may also be of the complement of a disclosed sequence or fragment thereof. The sequences in Table 1 include nucleotides from approximate positions 987 to 1135 of the HPV E1 region.

TABLE 1
Multiple Alignment of DNA sequences of part of E1 gene for high and
low risk HPV genotypes

| SEQ | |
|---|---|
| ID | 10 20 30 40 50 60 70 80 |
| NO: | ....\|....\|....\|....\|....\|....\|....\|....\|....\|....\|....\|....\|....\|....\|....\|....\| |
| 1 | CAGTGATACAGGTGAA---GATTTGGTAGATTTTATAGTAAATGATAATGATTATTTAACACAGGCAGAAACAGAGACAG |
| 2 | AACAGACACAGGGTCG---GATATGGTAGATTTTATTGATACACAAGGAACATTTTGTGAACAGGCAGAGCTAGAGACAG |
| 3 | TAGTGATACTGGGGAG---GATATGGTTGACTTTATTGACAATTGTAATGTATACAACAATCAGGCAGAAGCAGAGACAG |
| 4 | AGATGACAGTGGCACG---GATTTACTAGAGTTTATAGATGATTCTATGGAAAATAGTATACAGGCAGACACAGAGGCAG |
| 5 | CTGTGACAGGGGGGAG---GATATGGTGGACTTTATAAATGATACAGATATATTAAACATACAGGCAGAAACAGAGACAG |
| 6 | AACAGATACAGGTTCA---GACCTGGCAGACTTTATTGATGATTCCACAGATATTTGTGTACAGGCAGAGCGTGAGACAG |
| 7 | AACAGATACAGGGTCG---GATATGGTAGATTTTATTGACACACAATTATCCATTTGTGAACAGGCAGAGCAAGAGACAG |
| 8 | AGATGATACAGGATCT---GATTTAATAAACTTTATAGATAGTGAAACTAGTATTTGCAGTCAGGCGGAACAGGAGACAG |
| 9 | ATATGATAGTGGAACA---GATCTAATAGATTTTATAGATGATTCAAATATAAATAATGAACAGGCAGAACATGAGGCAG |
| 10 | GGATGAAATAGATACA---GATTTAGATGGATTTATAGACGATTCATATATACAAAATATACAGGCAGACGCAGAAACAG |
| 11 | AGACGATAGTGGTACA---GATTTAATAGAGTTTATAGATGATTCAGTACAAAGTACTACACAGGCAGAAGCAGAGGCAG |
| 12 | AACAGATACAGGTTCA---GACTTGGTAGATTTTATTGATGATACCACAACAATTTGTGTACAGGCAGAGCGCGAGACAG |
| 13 | AAATGATACAGGGTCT---GATATAATAGACTTTATAGATACAAATAACAGTATTTGCAGTCAGGCGGAACAAGAGACAG |
| 14 | GAATGAAACAGATACA---GATGTAGATGGATTTATAGACAATACACTTATAAACAATACACAGGAAGACAGGGAGACAG |
| 15 | AACAGATACAGGTTCA---GACATGGTAGATTTTATTGATGATTCTACACATATTTGTATACAGGCAGAGCGTGAGACAG |
| 16 | AAGC---ACCGAATCT---GATTTGGATGGGTTTATAGACAATAGTAATATAATATCTACACAGGCAGAAAGGGAGACAG |
| 17 | AGGGGATACAGATGAGTCGGAAATGGGGGATTTTATTGATAATGCACATATACCAAATATATATGCACAACAGGAAATTG |
| 18 | GGTGGAGGACAGTGGGTATGACATGGTGGACTTTATTGATGACAGCAATATTACACACAATTCACTGGAA---------G |
| 19 | GGTGGAGGACAGTGGGTATGACATGGTGGACTTTATTGATGACAGCAATATTACACACAATTCACTGGAA---------G |
| 20 | AGTGGAGGATAGTGGGTTGGATATGGTGGACTTTATAGATGACAGGCCTATTACACACAATTCCATGGAA---------G |
| 21 | AGAGGA---TAGTGGATTTGATATGATAGATTTTATTGATAATAGTGTTGTGGCAGAGGAACATGTAGAACTAAGTAATG |
| 22 | AGAGGAAGATAGTGGGTTTGATATGGTAGATTTTATTAATAATA---CATTAGAAGACAGTTGTACAGACCACAGCAGTG |
| 23 | TGTAGACGATAGTGGGTTAGATCTTGTGGATTTTGTAGATAATAGTACAGTAATACATACAAAGCAGGTAC------ATG |
| 24 | ---GGAGGACAGTGGGCTTGATATGGTGGACTTTATTGATAATAGTGTGTCACAGGTAGAGGGGCAGGAA------AATC |
| 25 | ---CATTGACACAGGGGAAGACCTAGTAGACTTCATAGATACAAGGCGCCCCGGGGATGGGCAGGAAGTGC--------- |
| 26 | AACAGATACAGGTTCA---GACTTGGCAGACTTTATTGATGATACTACAGATATTTGTGTACAGGCAGAGCGCGAGACAG |
| 27 | ---TGAGGACAGGGGAGAAGATCTGGTAGACTTTATAGACACAAGATCCTTAGGGGATGGGCAGGAAGTGC--------- |
| 28 | ---TGCAGATACAGGAGAGGATCTAGTAGATTTCATAGATACACGATATCCAGGGGATGGGCAGGAAGTGC--------- |
| SEQ | |
| ID | |
| NO: | 90 100 110 120 130 140 150 |
| | ....\|....\|....\|....\|....\|....\|....\|....\|....\|....\|....\|....\|....\|....\|... |
| 1 | CACATGCGTTGTTTACTGCACAGGAAGCAAAACAACATAGAGATGCAGTACAGGTTCTAAAACGAAAGTATTT |
| 2 | CACAGGCATTGTTCCATGCGCAGGAGGTCCACAATGATGCACAAGTGTTGCATGTTTTAAAACGAAAGTTTGC |
| 3 | CACAGGCATTGTTTCATGCACAGGAAGCGGAGGAACATGCAGAGGCTGTGCAGGTTCTAAAACGAAAGTATGT |
| 4 | CCCGGGCATTGTTTAATATACAGGAAGGGGAGGATGATTTAAATGCTGTGTGTGCACTAAAACGAAAGTTTGC |
| 5 | CACAAGCATTATTTCATGCACAGGAGGAGCAAACACACAAAGAGGCTGTACAGGTCCTAAAACGAAAGTATGC |
| 6 | CACAGGTACTTTTACATATGCAAGAGGCCCAAAGGGATGCACAAGCAGTGCGTGCCTTAAAACGAAAGTATAC |
| 7 | CACAGGCATTGTTCCATGCGCAGGAAGTTCAGAATGATGCACAGGTGTTGCATCTTTTAAAACGAAAGTTTGC |
| 8 | CACGGGCGTTGTTTCAGGCCCAAGAATTACAGGCAAACAAAGAGGCTGTGCATCAGTTAAAACGAAAGTTTCT |
| 9 | CCCGGGCATTGTTTAATGCACAGGAAGGGGAGGATGATTTACATGCTGTGTCTGCAGTAAAACGAAAGTTTAC |
| 10 | -TCAACAATTGTTGCAAGTACAAACAGCACATGCAGATAAACAGACGTTGCAAAAACTAAAACGAAAGTATAT |
| 11 | CCCGAGCGTTGTTTAATGTACAGGAAGGGGTGGACGATATAAATGCTGTGTGTGCACTAAAACGAAAGTTTGC |
| 12 | CACAGGCCTTGTTTAATGTGCAGGAAGCCCAAAGGGATGCACGGGAAATGCATGTTTTAAAACGAAAGTTTG- |
| 13 | CACGGGCGTTGTTTCAGGTCCAAGAAACACAGGCACACAAAGAGGCTGCACAGCATCTAAAACGAAAGTTTTT |
| 14 | CTCAACAATTATTGCAAGTACAAACAGCACATGCAGATGCACAGACGTTGCAAAAACTAAAACGAAAGTATAT |
| 15 | CACAGGTACTTTTGAATATGCAAGAGGCCCAAAGGGATGCACAAAGGGTGCGTGCCCTAAAACGAAAGTATAC |
| 16 | CTCAGCAGTTGTTACATGCCAAAACA-CACATGCAGATACACAGACGCTGCAGAAATTAAAACGAAAGTATTT |
| 17 | CACAGGCATTGTATCAGTCACAGCAAGCAAATGCAGACAATGAGGCTATACGTGTTCTAAAACGAAAGTTTAC |
| 18 | CACAGGCATTGTTTAACAGGCAGGAGGCGGACACCCATTATGCGACTGTGCAGGACCTAAAACGAAAGTATTT |
| 19 | CACAGGCATTGTTTAACAGGCAGGAGGCGGACACCCATTATGCGACTGTGCAGGACCTAAAACGAAAGTATTT |
| 20 | CACAGGCATTGTTAAACGAGCAGGAGGCGGATGCTCATTATGCGGCTGTGCAGGACCTAAAACGAAAGTATTT |
| 21 | CACAGGCACTTTTACATGTACAGCAGACATGTGCAGATGCTGCTGACCTGTGCGAGTTAAAACGAAAGTACAT |
| 22 | CGCAGGCTCTGTTAAATGCACAACAAGCGGATGCTGATGCTGCTATAGTGCAGGAGTTAAAACGAAAGTACAT |
| 23 | CACAAGCCTTATTAAATAAACAACAAGCACATGCAGATCAGGAGGCAGTACAGGCACTAAAACGAAAGCTATT |
| 24 | CACAGGCATTGTTACATGCCCAACAGCTGCAGGCAGATGTAGAGGCAGTGCAACAATTAAAACGAAAGTATAT |
| 25 | CGCTTGCGTTGTTCGTTCAACAAAATGCACAGGATGACGCTGCAACGGTGCAGGCACTAAAACGAAAGTATAC |
| 26 | CACAGGTACTGTATAATATGCAAGAGGCCCAAAGGGATGCACAATCAGTGCGTGCCTTAAAACGAAAGTATGG |
| 27 | CGTTAGATTTGTTCGTGCAACAAAATGCACGGGATGACGCTGCAACCGTGCAGGCCCTAAAACGAAAGTATAC |
| 28 | CGTTAGAACTGTTTGTTCAACAAAATGCACAGGATGACGCTGCAGCGGTGCATGCACTAAAACGAAAGTATAT |

Detection of HPV Sequences

The detection of one or more HPV sequences, including those disclosed herein, may be by the use of nucleic acid amplification as a non-limiting example. In some cases, the amplification is PCR-based. Optionally, the amplification includes use of one or more primers that contains one or more nucleotides, or sequences, that are not present in HPV such that the amplified molecule will contain a combination of sequences that is not naturally occurring in HPV. In some cases, the sequence is an adapter at the end of one or more primers. Alternatively, one or more of the primers contains an attached detectable label that is not present in HPV sequences. The disclosure includes a composition that is, or contains, one or more of these synthetic, non-naturally occurring, molecules. The disclosed fragments of the HPV genome are used as a marker for the detection of the high risk type viruses. Oligonucleotide primer and probe compositions that target this marker fragment are disclosed for use in detecting high risk HPV in a clinical urine sample.

In some embodiments, a PCR-based amplification includes use of 5′-CAGGCAGAATTAGAGRCAGC-3′, wherein R is A or G (SEQ ID No:29) and the primer is optionally labeled, as the forward primer and 5′-tccaccacaWACTTTCGTTTTA-3′, wherein W is T or A (SEQ ID 30) as the reverse primer. Lowercase nucleotides in the reverse primer are a selected tail to adjust the melting temperature (Tₘ) of the primer. Other random sequences may also be used. The expected size of the amplicon is 93-96 basepairs. In some cases, the forward primer is labeled and represented by 5′-FAM-CAGGCAGAATTAGAGRCAGC-3′ wherein R is A or G (SEQ ID No:31). These primer pairs detect high-risk forms of HPV.

In some cases, these primers have been applied to detect HPV types with sensitivities greater than 84%. For example, the detection of HPV types 16 and 18 may be accomplished with a sensitivity greater than 75%, greater than 80%, greater than 85%, greater than 90%, or greater than 95% as described herein. The primers may also be applied to detect HPV types 16, 18, 31, 33, 35, 39, 45, 51, 52, 56, 58, 59, and 68 with a sensitivity greater than 84%, greater than 86%, greater than 88%, greater than 90%, or greater than 92% as described herein. In further cases, the primers may be used to detect HPV types 31, 33, 35, 39, 45, 51, 52, 56, 58, 59, and 68 with a sensitivity greater than 84%, or to detect HPV types 31, 33, 45, 52, and 58 with a sensitivity greater than 75%, greater than 80%, greater than 82%, or greater than 84% as described herein.

In other embodiments, the amplifying includes a technique selected from hybridization; nested primer PCR; Real Time PCR; nucleic acid hybridization; Cyclic Probe Reaction; Single-Strand Conformation Polymorphism (SSCP); Strand Displacement Amplification (STA); and Restriction Fragment Length Polymorphism (RFLP). Nested PCR can be used to improve sensitivity by several orders of magnitude. In further embodiments, the amplifying may be followed by sequencing of the amplified material, such as sequencing of one or more amplified HPV sequences. In yet additional embodiments, the detection of HPV sequences may be performed by use of a sequencing primer that is sufficiently complementary and specific to HPV sequences to permit specific and direct sequencing without the need for prior amplification.

Additionally, the disclosed methods may further include quantifying the HPV nucleic acids by methods known to the skilled person.

When using PCR to detection an HPV sequence, the detecting step includes a PCR reaction comprising primer pairs sufficiently complementary to hybridize with an HPV sequence. Optionally, the sequence is within the E1 gene of HPV, such as with use of primer pairs sufficiently complementary to hybridize with a nucleic acid sequence represented by SEQ ID NOs: 1-28. In other embodiments, the detecting step includes a PCR reaction that uses primer pairs sufficiently complementary to hybridize with a nucleic acid sequence represented by SEQ ID NOs: 32-46 (as shown in Table 2) or a complementary sequence thereof.

TABLE 2
Individual oligonucleotides of high risk and
low-risk HPV types

| SEQ | | |
|---|---|---|
| ID | HPV | Nucleotide Sequence |
| 32 | 16 | CAGTGATACAGGTGAAGATTTGGTAGATTTTATAGTAAATGATAATGATTATTTA |
| | | ACACAGGCAGAAACAGAGACAGCACATGCGTTGTTTACTGCACAGGAAGCAAAA |
| | | CAACATAGAGATGCAGTACAGGTTCTAAAACGAAAGTATT |
| 33 | 18 | AACAGACACAGGGTCGGATATGGTAGATTTTATTGATACACAAGGAACATTTTGT |
| | | GAACAGGCAGAGCTAGAGACAGCACAGGCATTGTTCCATGCGCAGGAGGTCCAC |
| | | AATGATGCACAAGTGTTGCATGTTTTAAAACGAAAGTTTG |
| 34 | 31 | TAGTGATACTGGGGAGGATATGGTTGACTTTATTGACAATTGTAATGTATACAAC |
| | | AATCAGGCAGAAGCAGAGACAGCACAGGCATTGTTTCATGCACAGGAAGCGGAG |
| | | GAACATGCAGAGGCTGTGCAGGTTCTAAAACGAAAGTATG |
| 35 | 33 | AGATGACAGTGGCACGGATTTACTAGAGTTTATAGATGATTCTATGGAAAATAGT |
| | | ATACAGGCAGACACAGAGGCAGCCCGGGCATTGTTTAATATACAGGAAGGGGAG |
| | | GATGATTTAAATGCTGTGTGTGCACTAAAACGAAAGTTTG |
| 36 | 35 | CTGTGACAGGGGGGAGGATATGGTGGACTTTATAAATGATACAGATATATTAAAC |
| | | ATACAGGCAGAAACAGAGACAGCACAAGCATTATTTCATGCACAGGAGGAGCAA |
| | | ACACACAAAGAGGCTGTACAGGTCCTAAAACGAAAGTATG |
| 37 | 39 | AACAGATACAGGTTCAGACCTGGCAGACTTTATTGATGATTCCACAGATATTTGT |
| | | GTACAGGCAGAGCGTGAGACAGCACAGGTACTTTTACATATGCAAGAGGCCCAA |
| | | AGGGATGCACAAGCAGTGCGTGCCTTAAAACGAAAGTATA |
| 38 | 45 | AACAGATACAGGGTCGGATATGGTAGATTTTATTGACACACAATTATCCATTTGT |
| | | GAACAGGCAGAGCAAGAGACAGCACAGGCATTGTTCCATGCGCAGGAAGTTCAG |
| | | AATGATGCACAGGTGTTGCATCTTTTAAAACGAAAGTTTG |
| 39 | 51 | AGATGATACAGGATCTGATTTAATAAACTTTATAGATAGTGAAACTAGTATTTGC |
| | | AGTCAGGCGGAACAGGAGACAGCACGGGCGTTGTTTCAGGCCCAAGAATTACAG |
| | | GCAAACAAAGAGGCTGTGCATCAGTTAAAACGAAAGTTTC |
| 40 | 52 | ATATGATAGTGGAACAGATCTAATAGATTTTATAGATGATTCAAATATAAATAAT |
| | | GAACAGGCAGAACATGAGGCAGCCCGGGCATTGTTTAATGCACAGGAAGGGGAG |
| | | GATGATTTACATGCTGTGTCTGCAGTAAAACGAAAGTTTA |
| 41 | 56 | GGATGAAATAGATACAGATTTAGATGGATTTATAGACGATTCATATATACAAAAT |
| | | ATACAGGCAGACGCAGAAACAGTCAACAATTGTTGCAAGTACAAACAGCACATG |
| | | CAGATAAACAGACGTTGCAAAAACTAAAACGAAAGTATA |
| 42 | 58 | AGACGATAGTGGTACAGATTTAATAGAGTTTATAGATGATTCAGTACAAAGTACT |
| | | ACACAGGCAGAAGCAGAGGCAGCCCGAGCGTTGTTTAATGTACAGGAAGGGGTG |
| | | GACGATATAAATGCTGTGTGTGCACTAAAACGAAAGTTTG |
| 43 | 59 | AACAGATACAGGTTCAGACTTGGTAGATTTTATTGATGATACCACAACAATTTGT |
| | | GTACAGGCAGAGCGCGAGACAGCACAGGCCTTGTTTAATGTGCAGGAAGCCCAA |
| | | AGGGATGCACGGGAAATGCATGTTTTAAAACGAAAGTTTG |
| 44 | 68 | AAATGATACAGGGTCTGATATAATAGACTTTATAGATACAAATAACAGTATTTGC |
| | | AGTCAGGCGGAACAAGAGACAGCACGGGCGTTGTTTCAGGTCCAAGAAACACAG |
| | | GCACACAAAGAGGCTGCACAGCATCTAAAACGAAAGTTTT |
| 45 | 6 | GGTGGAGGACAGTGGGTATGACATGGTGGACTTTATTGATGACAGCAATATTACA |
| | | CACAATTCACTGGAAGCACAGGCATTGTTTAACAGGCAGGAGGCGGACACCCAT |
| | | TATGCGACTGTGCAGGACCTAAAACGAAAGTAT |
| 46 | 11 | GGTGGAGGACAGTGGGTATGACATGGTGGACTTTATTGATGACAGGCATATTACA |
| | | CAAAATTCTGTGGAAGCACAGGCATTGTTTAATAGGCAGGAGGCGGATGCTCATT |
| | | ATGCGACTGTGCAGGACCTAAAACGAAAGTAT |

In some embodiments, a disclosed amplification reaction uses one or more forward primers selected from SEQ ID NOs: 47-58 and 72-83. In other embodiments, a disclosed amplification reaction uses one or more reverse primers selected from SEQ ID NOs:59-71 and 84-88. Paired combinations of these forward and reverse primers may also be used. In some cases, a disclosed amplification reaction uses one or more primer pairs selected from SEQ ID NOs: 47 or 72 and 59 or 84, 48 or 73 and 60 or 85, 49 or 74 and 61, 50 or 75 and 62 or 86, 51 and 63, 52 or 76 and 64, 53 or 45 and 65, 54 or 79 and 67, 55 or 80 and 68 or 88, 56 or 81 and 69, 57 or 82 and 70, or 58 or 83 and 71.

TABLE 4

| SEQ | HPV | |
|---|---|---|
| ID | Type | Nucleotide Sequence |
| 47 | 16 | Forward caactccatctACACAGGCAGAAACAGAGACAG |
| 48 | 18 | Forward caactccatctGAACAGGCAGAGCTAGAGACAG |
| 49 | 31 | Forward caactccatctAATCAGGCAGAAGCAGAGACAG |
| 50 | 33 | Forward caactccatctATACAGGCAGACACAGAGGCAG |
| 51 | 35 | Forward caactccatctATACAGGCAGAAACAGAGACAG |
| 52 | 39 | Forward caactccatctGTACAGGCAGAGCGTGAGACAG |
| 53 | 45 | Forward caactccatctGAACAGGCAGAGCAAGAGACAG |
| 54 | 52 | Forward caactccatctGAACAGGCAGAACATGAGGCAG |
| 55 | 56 | Forward caactccatctATACAGGCAGACGCAGAAACAG |
| 56 | 58 | Forward caactccatctACACAGGCAGAAGCAGAGGCAG |
| 57 | 59 | Forward caactccatctGTACAGGCAGAGCGCGAGACAG |
| 58 | 68 | Forward caactccatctAGTCAGGCGGAACAAGAGACAG |
| 59 | 16 | Reverse TGCTTCCTGTGCAGTAAACAACGCATG |
| 60 | 18 | Reverse GACCTCCTGCGCATGGAACAATGC |
| 61 | 31 | Reverse CGCTTCCTGTGCATGAAACAATG |
| 62 | 33 | Reverse CCCTTCCTGTATATTAAACAATGCC |
| 63 | 35 | Reverse CTCCTCCTGTGCATGAAATAATGCTTG |
| 64 | 39 | Reverse GGCCTCTTGCATATGTAAAAGTAC |
| 65 | 45 | Reverse AACTTCCTGCGCATGGAACAATG |
| 66 | 51 | Reverse TAATTCTTGGGCCTGAAACAACG |
| 67 | 52 | Reverse CCCTTCCTGTGCATTAAACAATG |
| 68 | 56 | Reverse GTGCTGTTTGTACTTGCAACAATTG |
| 69 | 58 | Reverse CCCTTCCTGTACATTAAACAACG |
| 70 | 59 | Reverse GGCTTCCTGCACATTAAACAAGG |
| 71 | 68 | Reverse TGTTTCTTGGACCTGAAACAACG |
| 72 | 16 | Forward CAGGCAGAAACAGAGACAG |
| 73 | 18 | Forward CAGGCAGAGCTAGAGACAG |
| 74 | 31 | Forward CAGGCAGAAGCAGAGACAG |
| 75 | 33 | Forward CAGGCAGACACAGAGGCAG |
| 76 | 39 | Forward CAGGCAGAGCGTGAGACAG |
| 77 | 45 | Forward CAGGCAGAGCAAGAGACAG |
| 78 | 51 | Forward CAGGCGGAACAGGAGACAG |
| 79 | 52 | Forward CAGGCAGAACATGAGGCAG |
| 80 | 56 | Forward CAGGCAGACGCAGAAACAG |
| 81 | 58 | Forward CAGGCAGAAGCAGAGGCAG |
| 82 | 59 | Forward CAGGCAGAGCGCGAGACAG |
| 83 | 68 | Forward CAGGCGGAACAAGAGACAG |
| 84 | 16 | Reverse TGCTTCCTGTGCAGTAAACAACG |
| 85 | 18 | Reverse GACCTCCTGCGCATGGAACAATG |
| 86 | 33 | Reverse CCCTTCCTGTATATTAAACAATG |
| 87 | 35 | Reverse CTCCTCCTGTGCATGAAATAATG |
| 88 | 56 | Reverse TGCTGTTTGTACTTGCAACAATTG |

More generally, an oligonucleotide or complementary sequence with at least 50%, 55%, 60%, 65%, 70%, 75%, 80%, 85%, 90%, 95%, 100% homology or identity, or any percentage point in between may be used to detect HPV sequences in a biological or clinical urine sample. In some embodiments, the oligonucleotide contains a sequence found in the E1 or L1 region of HPV. In some cases, a pair of oligonucleotide primers may hybridize to binding sites which are either immediately adjacent to each other on the target sequence or slightly overlapping (having no intervening sequences between the primer binding sites). In some embodiments, oligonucleotides selected from regions of the E1 or L1 gene of HPV may be used to detect specific RNA transcripts of the E1 or L1 gene by a reverse transcription PCR reaction.

In certain embodiments of the disclosure, multiple pairs of primers are used in a reaction contained in single tube. Group or “multiplex” amplification reactions include 1-5, 5-10, 10-15, 15-20, 20-25 primers, or any number in between. Multiplex amplification reactions may be used to identify multiple HPV types present in a biological or clinical urine sample. For example, the primers listed in Tables 2 or 4 are applied to a given sample in the context of a single amplification reaction. In additional embodiments, the disclosed primers may optionally be used with appropriately selected oligonucleotide probes to detect HPV sequences.

The disclosure further provides detection of HPV sequences in additional modalities, such as (i) direct detection of the most frequent high risk types only using nucleic acid (NA) amplification or other analytical methods, (ii) direct detection of the most frequent high risk types using a two step process (NA amplification with a subsequent analysis of the product by hybridization or sequencing), and (iii) amplification and analysis of high and low risk HPV types in a single reaction.

Reagents, Kits and Uses

As described herein, the disclosure provides reagents and materials for the performance of the disclosed methods. The reagents and materials may be prepared, packaged or otherwise presented for use in one or more of the disclosed methods, such as for the detection of HPV nucleic acids or their preparation for analysis as non-limiting examples. In some embodiments, a reagent or material may be one for the collection or storage of a urine sample, for the isolation or preparation of HPV nucleic acids, or for the detection of an HPV nucleic acid. Non-limiting examples of such reagents include an agent to reduce nucleic acid degradation, such as EDTA and/or a chaotropic salt like GITC; a protease activity, such as proteinase K; a nucleic acid carrier agent, such as carrier RNA; binding, wash, and/or elution buffers, such as a Tris based buffer; an adsorbent material for nucleic acids, such as a silica-based adsorbent; a disclosed oligonucleotide, such as a primer or probe with sufficient homology to all or part of a region of the HPV genome; a pre-fabricated array or microarray; and a component of an amplification or hybridization reaction, such as a nucleotide triphosphate (e.g., dATP, dCTP, dGTP and/or dTTP) or DNA polymerase. In additional embodiments, a reagent or material may have an identifying description or label, while in other cases, a reagent or material may be packaged with a description or instructions relating to its use in one or more of the methods disclosed herein.

In further embodiments, a reagent or material is prepared or packaged as a kit produced in accordance with well known procedures. The disclosure thus provides kits comprising reagents or materials for performance of one or more of the disclosed methods. Such kits optionally include a reagent or material with an identifying description or label. In some cases, a kit includes instructions relating to using the kit in one or more of the disclosed methods. Such a kit may comprise containers, such as for the collection or transfer of a urine sample, or with one or more of the reagents and materials disclosed herein.

The disclosure further provides a use of one or more disclosed reagent or material in the preparation of a kit, assay, diagnostic, or medicament for performing one or more of the disclosed methods.

Having now generally provided the disclosure, the same will be more readily understood through reference to the following examples which are provided by way of illustration, and are not intended to be limiting of the disclosure, unless specified.

### EXAMPLES

### Example 1

### Clinical Samples

154 urine samples were obtained from a cohort of women who were referred to a colposcopy clinic. The subjects were identified based on one or more abnormal Papanicolaou tests (Pap smears). The women were not pregnant, had not been treated previously for cervical intraepithelial neoplasia (CIN), and did not undergo a hysterectomy. The population of subjects was expected to have a high prevalence (˜80%) of HPV (all types).

The samples were treated with addition of EDTA to approximately 40-70 mM final concentration before storage at reduced temperature. Some samples were frozen while others were stored at about 4° C.

### Example 2

### Sample Preparation

An aliquot of 500 μl of each urine sample was transferred to a tube. An equal amount of lysis buffer containing GITC (such as QIAGEN AL buffer) with about 11.2 μl/ml of carrier RNA is added to the sample followed by addition of an aliquot of protease. Proteolysis was allowed to proceed at approximately 56° C. for about 15 minutes. Ethanol (600 μl of 96-100%) was added for about 5 minutes at room temperature to enhance nucleic acid recovery.

The tube's contents were applied to a silica-based nucleic acid affinity column (such as the QIAmp MinElute column) and washed at least twice with ethanol rinses. Bound nucleic acids were eluted with 10 mM Tris, pH 8.0, optionally containing a preservative such as 0.04% azide.

### Example 3

### Sample Analysis (HPV Detection)

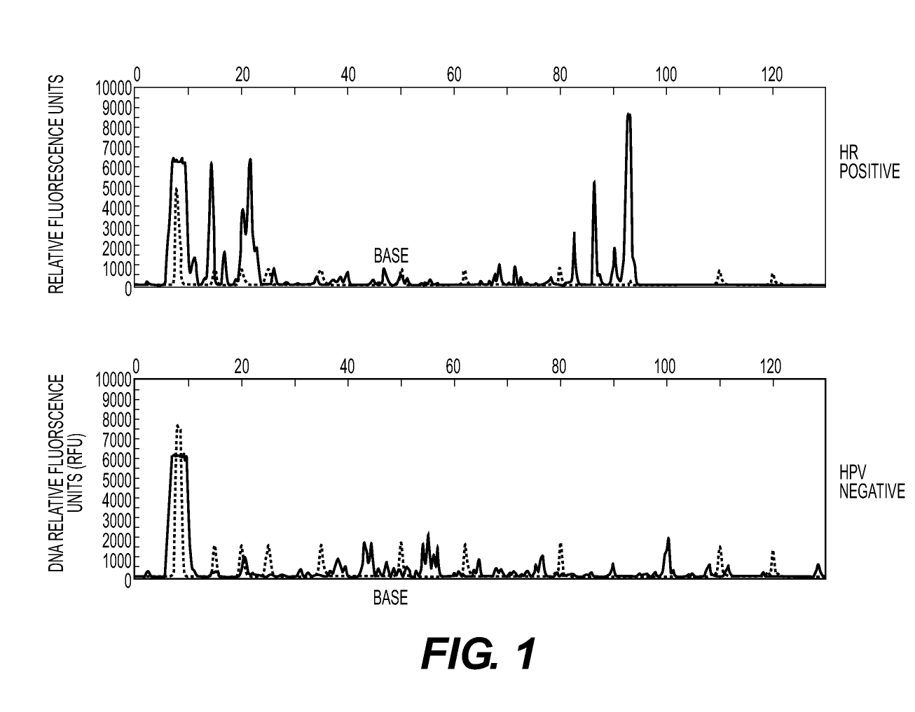

Prepared nucleic acids from each sample were PCR amplified with a forward and reverse primer pair (SEQ ID Nos: 30 and 31) followed by capillary electrophoresis to detect the presence of an amplicon of 93-96 bp—the expected size of a high-risk HPV PCR product. FIG. 1 shows exemplary traces from a “positive” (HPV 16) sample and a “negative” (HPV 54) sample. Positive and negative refer to whether the primer pair is complementary to, and so would amplify, an HPV sequence and as confirmed by sequencing described below. The trace represents FAM-labeled products, including any amplified material from the forward primer and unincorporated forward primer (large peak at approximately 15 bp).

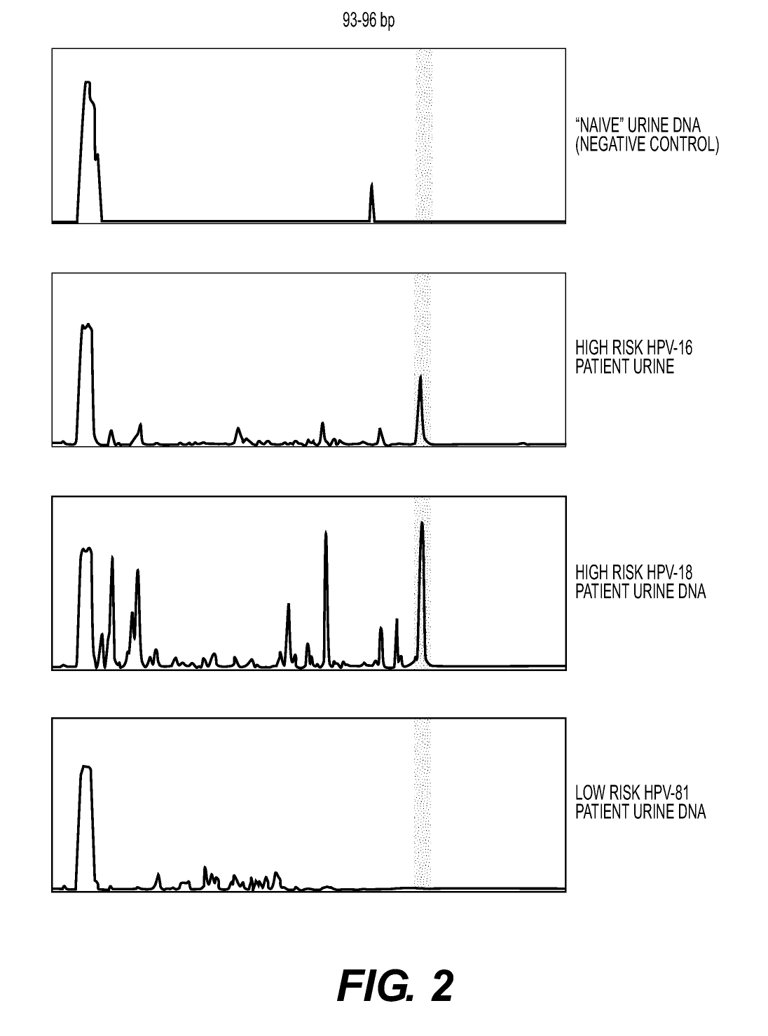

FIG. 2 shows exemplary traces from a negative control urine and two high risk urine samples (HPV 16 and 18) and one low risk sample (HPV 81). The identification of the HPV types was confirmed by sequencing as described below.

For fragment analysis, the instrumentation was a Genetic Analyzer ABI3130 and the Genetic Analyzer ABI 3130x1 with POP-7 polymer; both instruments are from Life Technologies (now Thermo Fisher). The Y-axis scale indicates relative fluorescence units. The conservative cutoff of 1200 relative fluorescence units was used to identify positive detection of an HPV sequence.

Summarized results from the 154 patient samples are compared to Bissett et al. (2011) as follows:

| | | | | Bissett et al. (2011) |
|---|---|---|---|---|
| HPV Type | Positive | Negative | Sensitivity | Sensitivity (p. 1747) |
| 16/18 | 89 | 2 | 97.8% | 75% |
| Any HR | 143 | 11 | 92.9% | 84% |
| Any HR | 54 | 9 | 85.7% | 83% |
| not 16/18 | | | | |
| 31/33/45/ | 45 | 8 | 84.9% | 75% |
| 52/58 | | | | |

HPV types 16 or 18 are indicated as “16/18”; HPV types 16, 18, 31, 33, 35, 39, 45, 51, 52, 56, 58, 59, and 68 are indicated as “Any HR”; and HPV types 31, 33, 45, 52, and 58 are indicated as “31/33/45/52/58.”

As seen from the comparison, the instant methods performed with a sensitivity higher than with the use of cell pellet material as reported by Bissett et al. (2011). The instant methods included the use of unfractionated urine and the presence of a nucleic acid carrier, which are not known to prevent inhibition of a PCR reaction.

### Example 4

### Data

The data from the 154 samples is presented in Table 5 below, with a one by shift in the instrumentation output to a range of 92-95 bp. A value of “9999” indicates an overscale output from the instrumentation but a confirmed positive trace after manual inspection. A value of “0” indicates no registered output from the instrumentation but a confirmed negative trace after manual inspection.

TABLE 5

| | Base Pair | Peak Height | Sanger |
|---|---|---|---|
| Sample | Size | (Rel. Fluor.) | Sequencing |
| 1 | 92.79 | 8696 | HPV-16 |
| 2 | 92.84 | 8805 | HPV-16 |
| 3 | 93.14 | 8731 | HPV-16 |
| 4 | 92.73 | 8759 | HPV-16 |
| 5 | 92.93 | 8379 | HPV-16 |
| 6 | 92.87 | 1462 | HPV-16 |
| 7 | 92.82 | 8409 | HPV-16 |
| 8 | 93.41 | 8402 | HPV-16 |
| 9 | 92.99 | 8659 | HPV-16 |
| 10 | 93.07 | 8708 | HPV-16 |
| 11 | 93.14 | 8718 | HPV-16 |
| 12 | 92.81 | 8619 | HPV-16 |
| 13 | 92.73 | 8295 | HPV-16 |
| 14 | 93.12 | 2702 | HPV-16 |
| 15 | 93.57 | 1245 | HPV-16 |
| 16 | 92.67 | 8365 | HPV-16 |
| 17 | 93.02 | 8624 | HPV-16 |
| 18 | 92.88 | 8706 | HPV-16 |
| 19 | 92.84 | 8401 | HPV-16 |
| 20 | 93.11 | 8659 | HPV-16 |
| 21 | 92.87 | 8604 | HPV-16 |
| 22 | 92.93 | 8942 | HPV-16 |
| 23 | | 9999 | HPV-16 |
| 24 | 93.03 | 8581 | HPV-16 |
| 25 | 93.09 | 8560 | HPV-16 |
| 26 | 93 | 8609 | HPV-16 |
| 27 | 92.94 | 8492 | HPV-16 |
| 28 | 92.81 | 8529 | HPV-16 |
| 29 | 92.89 | 8448 | HPV-16 |
| 30 | 93.78 | 7822 | HPV-16 |
| 31 | 92.94 | 8705 | HPV-16 |
| 32 | 93.06 | 8494 | HPV-16 |
| 33 | 93.04 | 8738 | HPV-16 |
| 34 | 92.92 | 320 | HPV-16 |
| 35 | 93.05 | 8401 | HPV-16 |
| 36 | 93.05 | 8578 | HPV-16 |
| 37 | 93.03 | 8885 | HPV-16 |
| 38 | 92.84 | 8380 | HPV-16 |
| 39 | 92.83 | 8436 | HPV-16 |
| 40 | 92.74 | 8335 | HPV-16 |
| 41 | 93.01 | 8079 | HPV-16 |
| 42 | 92.85 | 8603 | HPV-16 |
| 43 | 93.33 | 92 | HPV-16 |
| 44 | 92.19 | 8279 | HPV-16 |
| 45 | 93.11 | 8105 | HPV-16 |
| 46 | 92.77 | 8542 | HPV-16 |
| 47 | 92.86 | 8409 | HPV-16 |
| 48 | 92.83 | 8700 | HPV-16 |
| 49 | 93.03 | 8066 | HPV-16 |
| 50 | 92.88 | 8477 | HPV-16 |
| 51 | 93.17 | 8410 | HPV-16 |
| 52 | 92.93 | 1255 | HPV-16 |
| 53 | 92.78 | 8522 | HPV-16 |
| 54 | 92.73 | 8671 | HPV-16 |
| 55 | 93.02 | 7382 | HPV-16 |
| 56 | 92.26 | 8504 | HPV-16 |
| 57 | 93.33 | 8487 | HPV-16 |
| 58 | 92.94 | 8608 | HPV-16 |
| 59 | 92.79 | 8743 | HPV-16 |
| 60 | 93.09 | 8912 | HPV-16 |
| 61 | 92.8 | 8440 | HPV-16 |
| 62 | 92.9 | 8715 | HPV-16 |
| 63 | 92.95 | 8796 | HPV-16 |
| 64 | 93.09 | 8878 | HPV-16 |
| 65 | 93.28 | 8449 | HPV-16 |
| 66 | 92.58 | 8562 | HPV-16 |
| 67 | 92.99 | 8880 | HPV-16 |
| 68 | 92.98 | 8491 | HPV-16 |
| 69 | 92.96 | 8667 | HPV-16 |
| 70 | 92.87 | 8751 | HPV-16 |
| 71 | 93.24 | 8531 | HPV-16 |
| 72 | 93.03 | 3402 | HPV-16 |
| 73 | 93.17 | 8248 | HPV-16 |
| 74 | 92.96 | 8637 | HPV-16 |
| 75 | 93.15 | 8567 | HPV-18 |
| 76 | 93.55 | 8395 | HPV-18 |
| 77 | 93.37 | 8304 | HPV-18 |
| 78 | 93.5 | 5714 | HPV-18 |
| 79 | 93.73 | 3929 | HPV-18 |
| 80 | 93.36 | 8419 | HPV-18 |
| 81 | 93.52 | 8576 | HPV-18 |
| 82 | 93.29 | 8556 | HPV-18 |
| 83 | 93.18 | 8383 | HPV-18 |
| 84 | 92.66 | 6932 | HPV-18 |
| 85 | 93.34 | 8138 | HPV-18 |
| 86 | 92.62 | 8700 | HPV-18 |
| 87 | 93.46 | 6817 | HPV-18 |
| 88 | 93.3 | 8693 | HPV-18 |
| 89 | 93.47 | 8528 | HPV-18 |
| 90 | 93.39 | 7748 | HPV-18 |
| 91 | 93.35 | 8798 | HPV-18 |
| 92 | 93.32 | 8537 | HPV-31 |
| 93 | 93.71 | 7946 | HPV-31 |
| 94 | 93.68 | 8618 | HPV-31 |
| 95 | 93.48 | 8308 | HPV-31 |
| 96 | 93.57 | 8661 | HPV-31 |
| 97 | 93.8 | 8516 | HPV-31 |
| 98 | 93.31 | 8177 | HPV-31 |
| 99 | 93.68 | 8649 | HPV-31 |
| 100 | 93.26 | 8600 | HPV-31 |
| 101 | 93.78 | 8403 | HPV-31 |
| 102 | 93.37 | 8619 | HPV-31 |
| 103 | | 9999 | HPV-31 |
| 104 | 93.17 | 8499 | HPV-31 |
| 105 | 93.57 | 8622 | HPV-31 |
| 106 | 93.5 | 8593 | HPV-31 |
| 107 | 93.61 | 8217 | HPV-31 |
| 108 | 93.48 | 8335 | HPV-31 |
| 109 | 93.85 | 7894 | HPV-31 |
| 110 | 93.74 | 8626 | HPV-31 |
| 111 | 93.56 | 8513 | HPV-31 |
| 112 | 93.87 | 8506 | HPV-33 |
| 113 | 93.93 | 8346 | HPV-33 |
| 114 | 94.22 | 778 | HPV-33 |
| 115 | | 0 | HPV-33 |
| 116 | 93.64 | 7885 | HPV-33 |
| 117 | 94.9 | 94 | HPV-33 |
| 118 | 94.05 | 279 | HPV-33 |
| 119 | 93.67 | 3982 | HPV-33 |
| 120 | 93.47 | 8374 | HPV-33 |
| 121 | 94.16 | 8458 | HPV-33 |
| 122 | 93.99 | 8613 | HPV-33 |
| 123 | 94.2 | 493 | HPV-33 |
| 124 | 93.67 | 4143 | HPV-33 |
| 125 | 94.04 | 8402 | HPV-33 |
| 126 | | 0 | HPV-33 |
| 127 | 92.97 | 8665 | HPV-39 |
| 128 | 92.9 | 8575 | HPV-39 |
| 129 | 92.52 | 8567 | HPV-39 |
| 130 | 92.93 | 8598 | HPV-39 |
| 131 | 93.51 | 8705 | HPV-45 |
| 132 | 93.37 | 8391 | HPV-45 |
| 133 | 93.33 | 8674 | HPV-45 |
| 134 | 93.15 | 8318 | HPV-45 |
| 135 | 94.06 | 8704 | HPV-52 |
| 136 | 92.39 | 8258 | HPV-52 |
| 137 | 94.28 | 8287 | HPV-52 |
| 138 | 94.22 | 8538 | HPV-52 |
| 139 | 94.32 | 8618 | HPV-52 |
| 140 | 92.36 | 8638 | HPV-56 |
| 141 | 92.39 | 8635 | HPV-56 |
| 142 | 93.45 | 8838 | HPV-56 |
| 143 | 94.13 | 8298 | HPV-58 |
| 144 | 94.12 | 8560 | HPV-58 |
| 145 | 94.26 | 5198 | HPV-58 |
| 146 | 94.23 | 8557 | HPV-58 |
| 147 | 94.32 | 171 | HPV-58 |
| 148 | | 0 | HPV-58 |
| 149 | 93.03 | 5110 | HPV-58 |
| 150 | 94.36 | 8653 | HPV-58 |
| 151 | 94.54 | 8050 | HPV-58 |
| 152 | 92.84 | 8348 | HPV-68 |
| 153 | 92.93 | 8683 | HPV-68 |
| 154 | | 0 | HPV-68 |

### Example 5

### Sequencing

Nucleic acids from each of the 154 samples were amplified using primers MY09 (5′-CGTCCMARRGGAWACTGATC-3′; SEQ ID No: 89) and MY11 (5′-GCMCAGGGWCATAAYAATGG-3′; SEQ ID NO:90) and then sequenced by the Sanger reaction to identify the HPV type. The sequencing reactions were performed by SeqWright.

### REFERENCES

Bissett et al. (2011) “Human papillomavirus genotype detection and viral load in paired genital and urine samples from both females and males” J. Med. Virol. 83:1744-1751
Boom et al. (1990) “Rapid and simple method for purification of nucleic acids” J. Clin. Microbiol. 28:495-503
Enerly et al. (2013) “Monitoring human papillomavirus prevalence in urine samples: a review” Clin. Epidemiol. 5:67-79
Smits et al. (2005) “High prevalence of human papillomavirus infections in urine samples from human immunodeficiency virus-infected men” J. Clin. Microbiol. 43(12):5936-5939
Strauss et al. (1999) “Detection and typing of human papillomavirus DNA in paired urine and cervical scrapes” Eur. J. Epidemiol. 15:537-543

All references cited herein, including patents, patent applications, and publications, are hereby incorporated by reference in their entireties, whether previously specifically incorporated or not.

Having now fully described the inventive subject matter, it will be appreciated by those skilled in the art that the same can be performed within a wide range of equivalent parameters, concentrations, and conditions without departing from the spirit and scope of the disclosure and without undue experimentation.

While this disclosure has been described in connection with specific embodiments thereof, it will be understood that it is capable of further modifications. This application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

What is claimed is:

1. A method of detecting human papillomavirus (HPV) nucleic acids in a urine sample from a human subject, the method comprising:
(a) isolating HPV nucleic acids, from an unfractionated urine sample collected from a human subject, from the sample and in the presence of an added nucleic acid carrier agent, and
(b) detecting the isolated HPV nucleic acids.

2. The method of claim 1, wherein said isolating comprises contacting the sample with a proteolytic activity.

3. The method of claim 1, wherein said isolating comprises contacting the sample with a chaotropic salt.

4. The method of claim 1, wherein said detecting comprises assaying for a sequence of the E1 or L1 gene of HPV.

5. The method of claim 4, wherein said assaying comprises amplifying a sequence of the E1 or L1 gene of HPV.

6. The method of claim 5, wherein said amplifying comprises a forward primer with the sequence 5′-CAGGCAGAATTAGAGRCAGC (SEQ ID NO:29), wherein R is A or G and the forward primer is optionally labeled.

7. The method of claim 6, wherein said amplifying further comprises a reverse primer with the sequence 5′-TCCACCACAWACTTTCGTTTTA (SEQ ID NO:30), wherein W is T or A.

8. The method of claim 6, wherein the forward primer is XEN-HPV-FAM-F.

9. The method of claim 5, wherein said amplifying comprises a technique selected from the group consisting of hybridization, polymerase chain reaction (PCR); nested primer PCR; Real Time PCR; nucleic acid hybridization; Cyclic Probe Reaction; Single-Strand Conformation Polymorphism (SSCP); Strand Displacement Amplification (STA); and Restriction Fragment Length Polymorphism (RFLP).

10. The method of claim 1, wherein said isolating comprises precipitation or adsorption of HPV nucleic acids to a solid adsorbent material.

11. The method of claim 1, further comprising the step of quantifying said nucleic acids.

12. The method of claim 1, further comprising reducing nucleic acid degradation in the urine sample, prior to said isolating, by addition of an inhibitory agent to the urine sample or by an increase in the pH, salt concentration, or temperature of the urine sample.

13. The method of claim 12, wherein said inhibitory agent is selected from ethylenediaminetetraacetic acid (EDTA), guanidine-HCl guanidine isothiocyanate, N-lauroylsarcosine, and sodium dodecylsulphate.

14. The method of claim 1, wherein said detecting is with a sensitivity greater than 84%
